(19) United States
(12) Patent Application Publication
(10) Pub. No.: US 2024/0065530 A1
(43) Pub. Date: Feb. 29, 2024

(54) LENS UNIT, IMAGE PICKUP APPARATUS, ENDOSCOPE, AND METHOD OF MANUFACTURING LENS UNIT

(71) Applicant: OLYMPUS CORPORATION, Tokyo (JP)

(72) Inventor: Kensuke SUGA, Kamiina-gun (JP)

(73) Assignee: OLYMPUS CORPORATION, Tokyo (JP)

(21) Appl. No.: 18/387,318

(22) Filed: Nov. 6, 2023

Related U.S. Application Data
(63) Continuation of application No. PCT/JP2021/028089, filed on Jul. 29, 2021.

(51) Int. Cl. A61B 1/00

(52) U.S. Cl. CPC A61B 1/00096; A61B 1/0011

(57) ABSTRACT

A lens unit includes a first optical element including a first principal surface that is an incident surface; and including a first glass substrate and a first resin, the first glass substrate including a frame-shaped cutout at an outer edge of the first principal surface, the first resin being disposed only in the cutout.

### CROSS REFERENCE TO RELATED APPLICATION

This application is a continuation application of PCT/JP2021/028089 filed on Jul. 29, 2021, the entire contents of which are incorporated herein by this reference.

### BACKGROUND OF THE INVENTION

### 1. Field of the Invention

The present invention relates to: a lens unit including a hybrid lens element in which a resin lens is disposed on a glass substrate; an image pickup apparatus including a lens unit including a hybrid lens element; an endoscope including an image pickup apparatus including a lens unit including a hybrid lens element; and a method of manufacturing a lens unit including a hybrid lens element in which a resin lens is disposed on a glass substrate.

### 2. Description of the Related Art

It is important that a lens unit of an image pickup apparatus installed at a distal end portion of an endoscope is reduced in diameter in order to be less invasive.

International Publication No. 2017/203592 discloses a lens unit that is a wafer level stacked body that can allow a lens unit with a small diameter to be efficiently manufactured. The wafer level stacked body is manufactured by cutting a stacked wafer in which a plurality of element wafers, each including a plurality of lens elements, are stacked with an adhesive layer in between.

### SUMMARY OF THE INVENTION

Embodiments of the present invention have an object of providing: a lens unit that is easy to manufacture and has high reliability; an image pickup apparatus that is easy to manufacture and has high reliability; an endoscope that is easy to manufacture and has high reliability; and a method of easily manufacturing a lens unit that has high reliability.

### Means for Solving the Problem

A lens unit of an embodiment includes a first optical element including a first principal surface that is an incident surface; a second principal surface on a side opposite to the first principal surface; and four first side surfaces, and including a first glass substrate including a frame-shaped cutout at an outer edge of the first principal surface; and a first resin disposed only in the cutout.

An image pickup apparatus of an embodiment includes a lens unit and an image pickup unit, the lens unit including a first optical element including a first principal surface that is an incident surface; a second principal surface on a side opposite to the first principal surface; and four first side surfaces, and including a first glass substrate including a frame-shaped cutout at an outer edge of the first principal surface; and a first resin disposed only in the cutout.

An endoscope of an embodiment includes an image pickup apparatus including a lens unit and an image pickup unit, the lens unit including a first optical element including a first principal surface that is an incident surface; a second principal surface on a side opposite to the first principal surface; and four first side surfaces, and including a first glass substrate including a frame-shaped cutout at an outer edge of the first principal surface; and a first resin disposed only in the cutout.

A method of manufacturing a lens unit of an embodiment includes: providing a first principal surface of a first element wafer with a plurality of grooves, having a first width, in a grid pattern, the first element wafer including a glass wafer as a base, the glass wafer including the first principal surface that is an incident surface and a second principal surface on a side opposite to the first principal surface; filling the grooves with a first resin; and cutting from the second principal surface along a plurality of grooves with a dicing blade including a cutting area with a second width narrower than the first width, in a state in which the first principal surface of a stacked wafer is fixed to a holding member, the stacked wafer including the first resin and the first element wafer.

### BRIEF DESCRIPTION OF THE DRAWINGS

FIG. 1 is a perspective view of an image pickup apparatus (lens unit) of a first embodiment;

FIG. 2 is a cross-sectional view taken along a line II-II in FIG. 1;

FIG. 3 is a flowchart of a method of manufacturing the image pickup apparatus of the first embodiment;

FIG. 4 is a perspective exploded view for explaining the method of manufacturing the image pickup apparatus of the first embodiment;

FIG. 5 is a cross-sectional view for explaining the method of manufacturing the image pickup apparatus of the first embodiment;

FIG. 6 is a cross-sectional view for explaining the method of manufacturing the image pickup apparatus of the first embodiment;

FIG. 7 is a cross-sectional view for explaining the method of manufacturing the image pickup apparatus of the first embodiment;

FIG. 8A is a cross-sectional view for explaining an image pickup apparatus of Modification 1 of the first embodiment;

FIG. 8B is a cross-sectional view for explaining an image pickup apparatus of Modification 2 of the first embodiment;

FIG. 9 is a perspective view of an image pickup apparatus of a second embodiment;

FIG. 10 is a cross-sectional view taken along a line X-X in FIG. 9;

FIG. 11 is a cross-sectional view for explaining a method of manufacturing the image pickup apparatus of the second embodiment;

FIG. 12 is a cross-sectional view for explaining the image pickup apparatus of the second embodiment; and

FIG. 13 is a perspective view of an endoscope of a third embodiment.

### BEST MODE FOR CARRYING OUT THE INVENTION

### First Embodiment

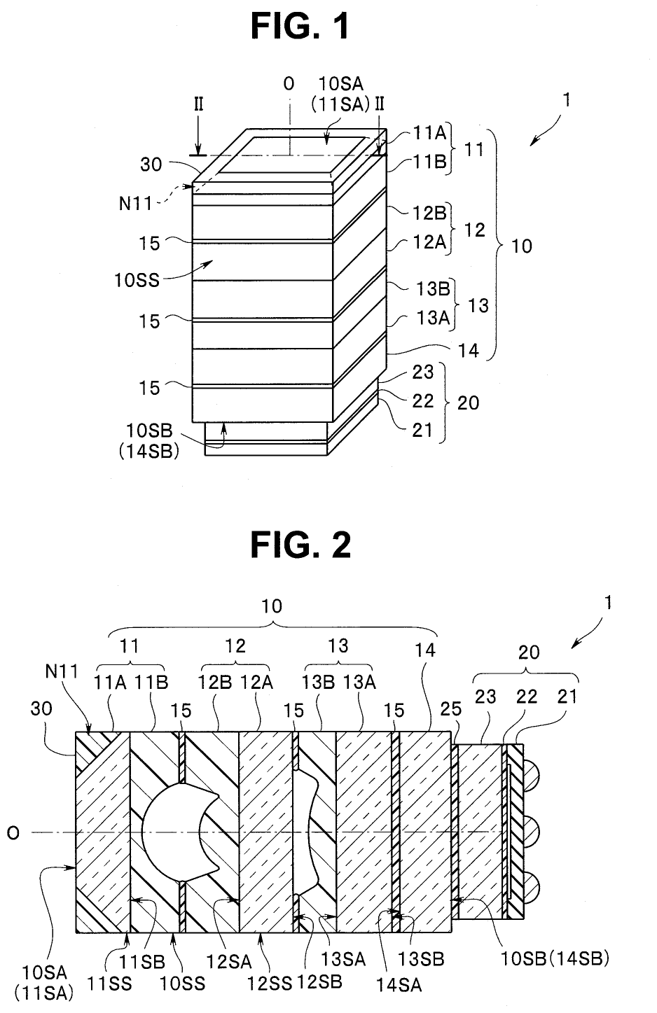

As shown in FIGS. 1 and 2, an image pickup apparatus 1 of the present embodiment includes a lens unit 10 and an image pickup unit 20. A reference character O indicates an optical axis of the lens unit 10. The image pickup unit 20 receives the object image obtained by concentrating the light by the lens unit 10 and converts the image into an image pickup signal.

Note that in the following description, drawings based on each embodiment are schematic. The relationship between the thickness and width of each part, the ratio of the thickness of each part, the relative angle, etc. differ from the actual configuration. There are parts included, dimensional relationships and ratios of which differ from each other among the drawings. Illustrations of some components are omitted.

The lens unit 10 has an incident surface 10SA and an emission surface 10SB on the side opposite to the incident surface 10SA. The lens unit 10 includes a first optical element 11 having the incident surface 10SA, a second optical element 12, and a third optical element 13, and a fourth optical element 14 having an emission surface 10SB. The first optical element 11, the second optical element 12, the third optical element 13, and the fourth optical element 14 are stacked in the order, and have principal surfaces having substantially the same sizes.

The first optical element 11 has a first glass substrate 11A as a base, the first glass substrate 11A having a first principal surface 11SA, a second principal surface 11SB on the side opposite to the first principal surface 11SA, and four first side surfaces 11SS. The first optical element 11, the first principal surface 11SA of which is the incident surface 10SA, is a hybrid lens element having a concave lens that is a resin lens 11B on the second principal surface 11SB.

There is a frame-shaped cutout N11 at an outer edge of the first principal surface 11SA. A first resin 30 is disposed only in the cutout N11.

The second optical element 12 has a second glass substrate 12A as a base, the second glass substrate 12A having a third principal surface 12SA, a fourth principal surface 12SB on the side opposite to the third principal surface 12SA, and four second side surfaces 12SS. The third principal surface 12SA is arranged to face the second principal surface 11SB. The second optical element 12 is a hybrid lens element having a convex lens 12B made of resin on the third principal surface 12SA.

The third optical element 13 has a third glass substrate 13A as a base, the third glass substrate 13A having a fifth principal surface 13SA and a sixth principal surface 13SB on the side opposite to the fifth principal surface 13SA. The fifth principal surface 13SA is arranged to face the fourth principal surface 12SB. The third optical element 13 is a hybrid lens element having a convex lens 13B made of resin on the fifth principal surface 13SA.

The fourth optical element 14 has a seventh principal surface 14SA and an eighth principal surface 14SB on the side opposite to the seventh principal surface 14SA. The eighth principal surface 14SB is the emission surface 10SB. The fourth optical element 14 is a filter element that removes unnecessary infrared rays (for example, light with a wavelength of 700 nm or more). The fourth optical element 14 may be a filter element in which a multilayer filter is disposed on a glass substrate. Note that although the description has been provided that the fourth optical element 14 is a filter element in which a filter is disposed, the fourth optical element may be omitted and the first to third optical elements may have a filter function instead.

The first glass substrate 11A, the second glass substrate 12A, and the third glass substrate 13A are made of, for example, borosilicate glass, quartz glass, or sapphire glass.

The first optical element 11 and the second optical element 12, the second optical element 12 and the third optical element 13, and the third optical element 13 and the fourth optical element 14 are each bonded by an adhesive layer 15 made of resin.

Note that the configuration of the lens unit of the present invention is not limited to the configuration of the lens unit 10, and is set according to specifications. For example, the lens unit may have not only lens elements but also spacer elements and aperture layers that define the distance between the lenses.

The image pickup unit 20 is bonded to the emission surface 10SB (eighth principal surface 14SB) of the lens unit 10 with an adhesive layer 25. The image pickup unit 20 has a cover glass 23 bonded to an image pickup device 21 with an adhesive layer 22. The image pickup device 21 is a CMOS (complementary metal oxide semiconductor) light receiving element or a CCD (charge coupled device).

In cutting a stacked wafer including a hybrid lens element in which a resin lens is disposed on a glass substrate, there have been cases in which chipping (cracking) occurs in the glass substrate. In particular, chipping is likely to occur on the principal surface of the glass substrate that is pasted to the dicing tape and is cut last. In the lens unit 10, the first principal surface 11SA of the first glass substrate 11A is the principal surface that has been cut last.

In the lens unit 10, there is a frame-shaped cutout N11 at the outer edge of the first principal surface 11SA, and a first resin 30 is disposed only in the cutout N11. The first resin 30 prevents the first glass substrate 11A from chipping occurrence, so that the lens unit 10 (image pickup apparatus 1) is easy to manufacture and has high reliability.

### <Manufacturing Method>

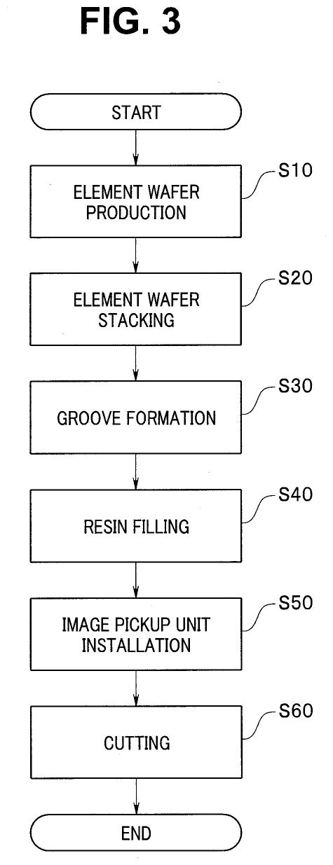

A method of manufacturing the image pickup apparatus 1 (lens unit 10) will be explained according to the flowchart in FIG. 3.

The lens unit 10 is a wafer level lens unit manufactured by cutting a stacked wafer, which is produced by stacking a plurality of element wafers each having a plurality of optical elements disposed in a matrix pattern. Note that the following describes the method of manufacturing the image pickup apparatus 1 by cutting a stacked wafer in which a plurality of image pickup units 20 are installed, as an example.

### <Step S10> Element Wafer Production

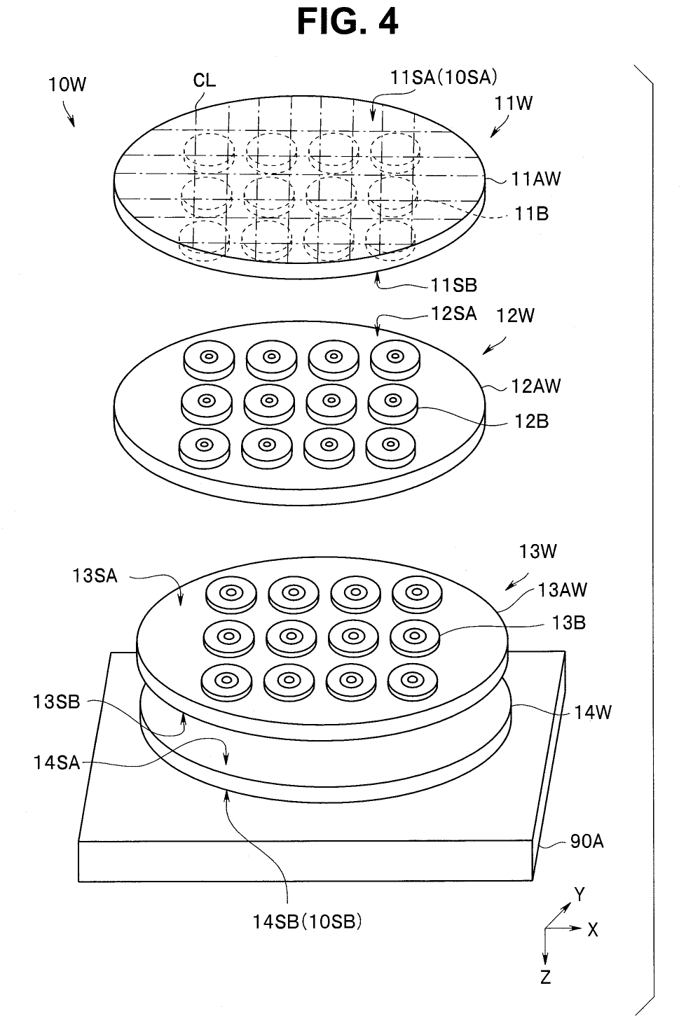

A plurality of element wafers 11W, 12W, and 13W shown in FIG. 4 are produced.

The first element wafer 11W including a plurality of the first optical elements 11 is produced by disposing a plurality of the resin lenses 11B on the second principal surface 11SB of a glass wafer 11AW. Note that the reference character CL is a cutting line in the cutting step (S60) to be described below. It is preferable to use energy-curable resin for the resin lens 11B.

Energy-curable resin receives external energy such as heat, ultraviolet rays, or electron beams, causing progress of crosslinking reaction or polymerization reaction. Energy-curable resins are made of, for example, transparent ultraviolet-curable silicone resins, epoxy resins, and acrylic resins. Note that “transparent” means that the material has low light absorption and low scattering to the extent that the material can withstand use in the wavelength range to be used.

The resin lens 11B is produced through a molding method in which resin is cured by irradiating the resin with ultraviolet rays, in a state in which the uncured liquid or gel-like resin is disposed on a glass wafer 11AW and a mold having a recessed portion with a predetermined inner surface shape is pressed against the resin. Note that in order to improve the interfacial adhesion strength between the glass and the resin, it is preferable to perform a silane coupling treatment or the like on the glass wafer before the resin is disposed. An element wafer 12W having a glass wafer 12AW as a base and an element wafer 13W having a glass wafer 13AW as a base are produced through methods similar to the method in the first element wafer 11W. An element wafer 14W is a filter wafer.

Since the outer shape of the resin lens manufactured by using the molding method is transferred from the inner surface shape of the mold, it is possible to easily produce a configuration having an outer edge portion that also serves as a spacer, and an aspherical lens.

### <Step S20> Element Wafer Stacking

For example, the adhesive layer 15 is disposed on the resin lens 11B of the first element wafer 11W through a transfer method. The adhesive layer 15 may be disposed through an inkjet method, for example. The adhesive layer 15 is, for example, a heat-curable epoxy resin. The adhesive layer 15 may be, for example, a light-shielding layer containing particles with light-shielding properties. A stacked wafer 10W is produced by stacking and bonding a plurality of the element wafers 11W, 12W, 13W, and 14W.

### <Step S30> Groove Formation

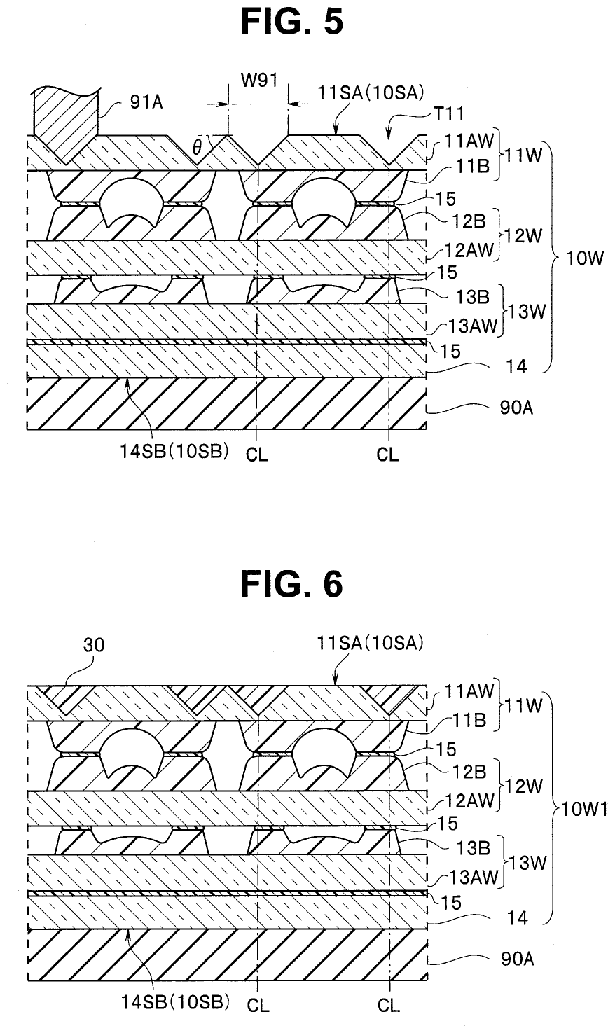

As shown in FIG. 5, the emission surface 10SB (eighth principal surface 14SB) of the stacked wafer 10W is fixed to a first holding member such as a first dicing tape 90A. With a first dicing blade 91A, a plurality of grooves T11 are formed in a grid pattern along the cutting line CL on the incident surface (first principal surface 11SA) of the stacked wafer 10W. The groove T11 is a V-groove with an opening width (first width) W91. For example, the groove T11 is formed by using a double-edged (V-shaped) first dicing blade 91A with a tip surface having an angle of 90 degrees. Note that the groove T11 may be formed up to the resin lens 11B. The angle θ of the groove T11 with respect to the first principal surface 11SA is, for example, 40 to 50 degrees.

### <Step S40> First Resin Filling

As shown in FIG. 6, the first resin 30 is disposed in the grooves T11 of the stacked wafer 10W and the first resin 30 undergoes a cure treatment, so that a stacked wafer 10W1 is produced. For example, the heat-curable first resin 30 is BCB (benzocyclobutene) resin, epoxy resin, or silicone resin. The first resin 30 is preferably a light-shielding resin containing particles with light-shielding properties, for example. The first resin 30 with light-shielding properties does not transmit light, and there is no risk of external light entering the optical path.

For example, a film is pasted on the incident surface 10SA before the groove forming step and then the grooves T11 are formed with the first dicing blade 91A, so that openings are formed in the film. The first resin 30 is applied over the film and then the film is detached, so that the first resin 30 is disposed only in the grooves T11.

After the first resin 30 is applied to the first principal surface 11SA in which the grooves T11 are formed, the first resin 30 may be removed in regions other than regions having the grooves T11 filled with the resin 30 by means of oxygen plasma treatment, polishing treatment, or the like.

### <Step S50> Image Pickup Unit Installation

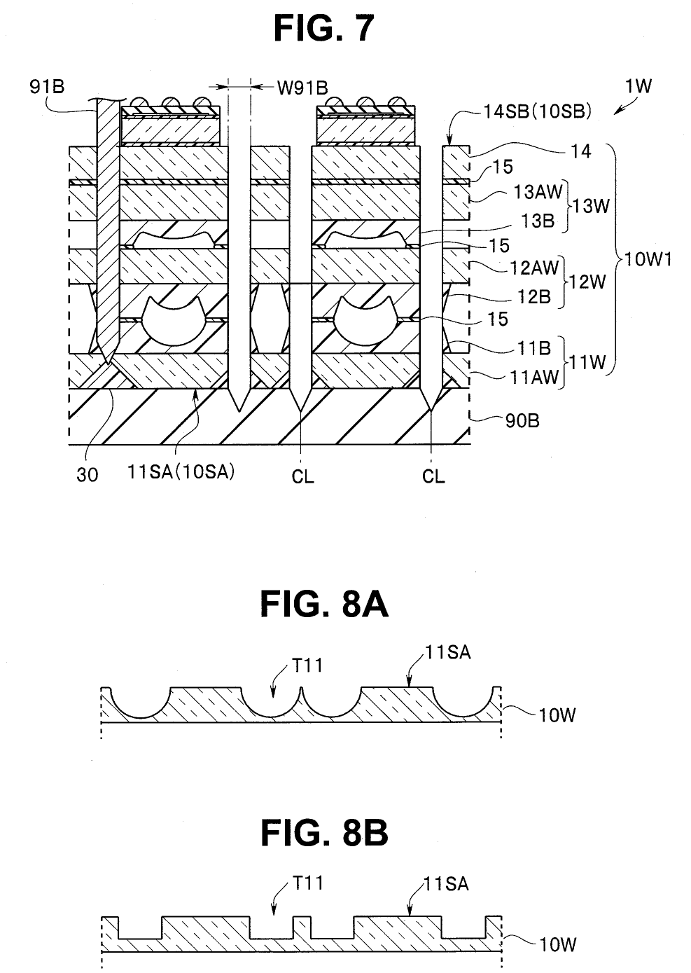

The stacked wafer 10W1 is removed from the first dicing tape 90A. Then, as shown in FIG. 7, the stacked wafer 10W1, which has been turned upside down, has the incident surface 10SA (first principal surface 11SA) fixed to a second holding member such as a second dicing tape 90B. Then, a plurality of image pickup units 20 are bonded to the emission surface 10SB (eighth principal surface 14SB) with the adhesive layer 25, so that a stacked wafer 1W is produced.

The image pickup units 20 are manufactured by cutting an image pickup device wafer formed by bonding a glass wafer which becomes a plurality of pieces of cover glass 23 to an element wafer including a plurality of image pickup devices 21, with an adhesive layer 22. Note that the stacked wafer 1W may be produced by bonding an image pickup wafer to the stacked wafer 10W1.

### <Step S60> Cutting

The stacked wafer 1W is cut with the second dicing blade 91B along the cutting line CL centered on a plurality of the grooves TI 1, in a grid pattern, filled with the first resin 30, to be segmented into the image pickup apparatuses 1.

The second width that is the width of the cutting area W91B of the second dicing blade 91B is smaller than the first width W91 of the groove T11. Therefore, as already explained, the lens unit 10 has a frame-shaped cutout N11 at the outer edge of the incident surface 10SA where chipping is most likely to occur, and the first resin 30 is disposed only in the cutout N11. The first resin 30 prevents the first glass substrate 11A from chipping occurrence, so that the lens unit 10 (image pickup apparatus 1) is easy to manufacture and has high reliability.

The image pickup apparatus 1 may be produced by installing the image pickup unit 20 on the lens unit 10 manufactured by cutting the stacked wafer 10W1.

Each groove T11 formed in the first principal surface 11SA of the stacked wafer 10W is not limited to a V-groove. For example, the groove of Modification 1 shown in FIG. 8A has a semicircular cross section, and the groove of Modification 2 shown in FIG. 8B has a rectangular cross section. The grooves may be formed by etching instead of using a dicing blade.

### Second Embodiment

A lens unit of the present embodiment is similar to the lens unit 10 and has the same effects, so components with the same functions are given the same reference numerals and characters and explanations will be omitted.

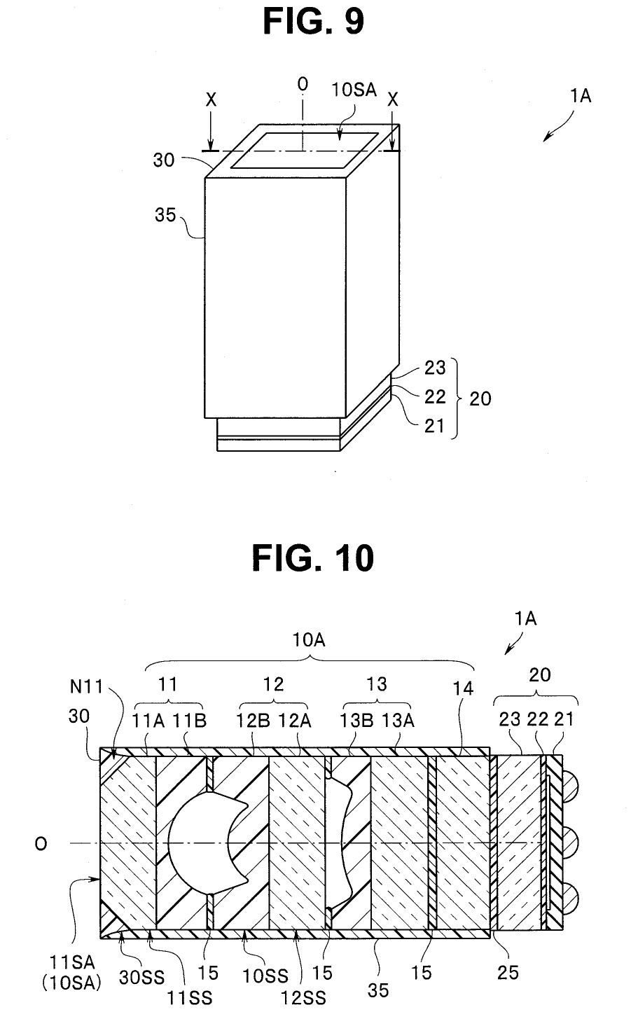

As shown in FIGS. 9 and 10, the lens unit 10A of the image pickup apparatus 1A of the present embodiment has the first resin 30, disposed only in the cutout N11 of the first glass substrate 11A, that protrudes from the four side surfaces 10SS. A second resin 35 is disposed on the side surfaces 10SS including the first side surfaces 11SS and the second side surfaces 12SS, and on the side surfaces 30SS of the first resin 30.

The outer dimension of the first resin 30 in the direction perpendicular to the optical axis at the incident surface 10SA is the same as the outer dimension of the second resin 35 in the direction perpendicular to the optical axis at the emission surface 10SB.

The lens unit 10A has higher mechanical strength than the lens unit 10 due to the second resin 35. Further, use of a light-shielding resin as the second resin 35 causes the lens unit 10A to prevent external light from entering the optical path and prevent light from leaking from the optical path.

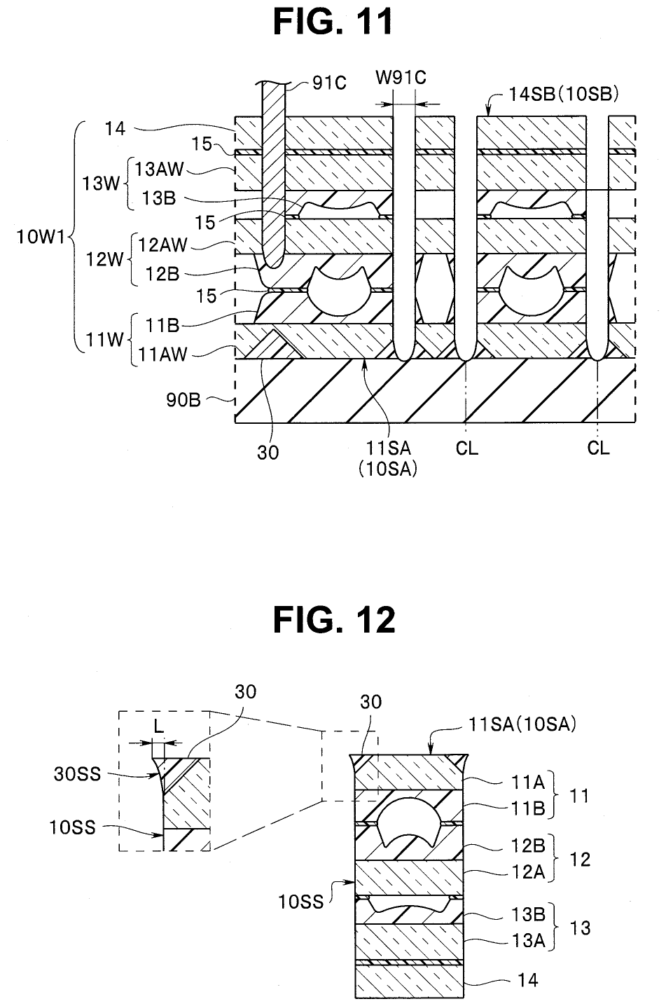

As shown in FIG. 11, the lens unit 10A uses a third dicing blade 91C, the tip surface of which has a curved cross section in the direction parallel to the long axis, in cutting the stacked wafer 10W1. Then, the stacked wafer 10W1 is cut so that the side surfaces 30SS, which are the cut surfaces of the first resin 30, protrude from the side surfaces 10SS. In other words, cutting is completed when the tip of the third dicing blade 91C reaches the second dicing tape 90B. The dicing blade 91C may have a V-shaped tip.

In the method of manufacturing the image pickup apparatus 1A, the image pickup unit 20 is bonded to the segmented lens unit 10A.

Although not shown, the second resin 35 is disposed on the side surfaces 10SS of the segmented lens unit 10A and the side surfaces 30SS of the first resin 30. Since the side surfaces 30SS of the first resin 30 protrude from the side surfaces 10SS, there is no risk of the second resin 35 being disposed also on the incident surface 10SA in disposing the second resin 35.

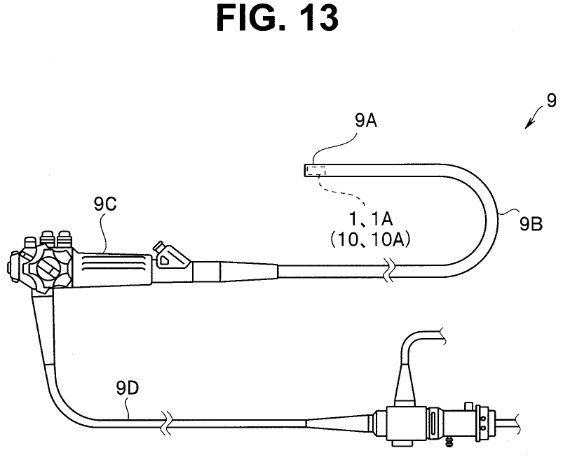

The protrusion length L of the first resin 30 from the side surface 10SS shown in FIG. 12 is, for example, more than 50 m and less than 400 μm, preferably more than 100 μm and less than 200 sm. The protrusion length L is the thickness of the second resin 35. If the protrusion length L exceeds the lower limit, the effect of the second resin 35 is significant. If the protrusion length L is less than the upper limit, the outer dimensions of the lens unit can be made less than the specification value.

### Third Embodiment

As shown in FIG. 13, an endoscope 9 of the present embodiment includes: a distal end portion 9A; an insertion portion 9B extending from the distal end portion 9A; an operation portion 9C installed on the proximal end side of the insertion portion 9B; and a universal cord 9D extending from operation portion 9C.

An image pickup apparatus 1 (1A) including a lens unit 10 (10A) is installed at the distal end portion 9A. An image pickup signal outputted from the image pickup apparatus 1 is transmitted to a processor (not shown) via a cable passing through the universal cord 9D. Further, a drive signal from the processor to the image pickup apparatus 1 is also transmitted via a cable passing through the universal cord 9D.

As described, the lens unit 10 (10A) is easy to manufacture and has high reliability. Therefore, the endoscope 9 is easy to manufacture and has high reliability.

The present invention is not limited to the embodiments and the like described above, and various modifications, combinations, and applications can be made without departing from the spirit of the invention.

What is claimed is:

1. A lens unit comprising:
a first optical element including a first glass substrate having a first principal surface that is an incident surface, a second principal surface on a side opposite to the first principal surface, and four first side surfaces, and including a frame-shaped cutout at an outer edge of the first principal surface, and a first resin disposed only in the cutout.

2. The lens unit according to claim 1, further comprising:
a second optical element including a second glass substrate including a third principal surface, a fourth principal surface on a side opposite to the third principal surface, and four second side surfaces, the third principal surface being arranged to face the second principal surface; and
an adhesive layer bonding the first optical element and the second optical element.

3. The lens unit according to claim 2, wherein at least one of the first optical element or the second optical element is a hybrid lens element including a resin lens.

4. The lens unit according to claim 2, wherein the first resin has light-shielding properties.

5. The lens unit according to claim 2, wherein the first resin protrudes from the first side surfaces.

6. The lens unit according to claim 5, further comprising a second resin covering side surfaces of the first resin, the first side surfaces, and the second side surfaces.

7. The lens unit according to claim 6, wherein the second resin has light-shielding properties.

8. An image pickup apparatus comprising:
a lens unit and an image pickup unit,
the lens unit including a first optical element including a first glass substrate having a first principal surface that is an incident surface, a second principal surface on a side opposite to the first principal surface, and four first side surfaces, and including a frame-shaped cutout at an outer edge of the first principal surface, and a first resin disposed only in the cutout.

9. An endoscope comprising:
an image pickup apparatus including a lens unit and an image pickup unit,
the lens unit including a first optical element including a first glass substrate having a first principal surface that is an incident surface, a second principal surface on a side opposite to the first principal surface, and four first side surfaces, and including a frame-shaped cutout at an outer edge of the first principal surface, and a first resin disposed only in the cutout.

10. A method of manufacturing a lens unit, comprising:
providing a first principal surface of a first element wafer with a plurality of grooves, having a first width, in a grid pattern along cutting lines, the first element wafer including a glass wafer as a base, the glass wafer including the first principal surface that is an incident surface and a second principal surface on a side opposite to the first principal surface;
filling the grooves with a first resin; and
cutting from the second principal surface along the plurality of grooves with a dicing blade including a cutting area with a second width narrower than the first width, in a state in which the first principal surface of a stacked wafer is fixed to a holding member, the stacked wafer including the first resin and the first element wafer.

11. The method of manufacturing the lens unit according to claim 10, further comprising:
forming the first resin so as to protrude from cut surfaces; and
disposing a second resin on side surfaces of the first resin and the cut surfaces after cutting with the dicing blade.
